(19) United States
(12) Patent Application Publication
(10) Pub. No.: US 2005/0267029 A1
(43) Pub. Date: Dec. 1, 2005

(54) Compounds which modulate amyloidogenesis and methods for their identification and use

(76) Inventors: John B. Ancsin, Kingston (CA); Elena Elimova, St. John's (CA); Robert Kisilevsky, Kingston (CA)

Correspondence Address: Licata & Tyrrell P.C., 66 East Main Street, Marlton, NJ, 08053, US

(21) Appl. No.: 11/098,674

(22) Filed: Apr. 4, 2005

Related U.S. Application Data
(60) Provisional application No. 60/559,122, filed on Apr. 2, 2004.

(51) Int. Cl. A61K 38/17; C12Q 1/68; C12N 5/08

(52) U.S. Cl. 514/12; 435/368

(57) ABSTRACT

A cell culture system for amyloidogenesis and methods for use of this cell culture system in identifying amyloid modulating compounds are provided. Also provided are compounds and methods for modulating the interaction of an amyloid polypeptide and heparan sulfate and for treating amyloid-associated diseases.

This patent application claims the benefit of priority from U.S. Provisional Application Ser. No. 60/559,122 filed Apr. 2, 2004 which is herein incorporated by reference in its entirety.

### FIELD OF THE INVENTION

The present invention relates to a cell culture system that transforms the acute-phase protein, serum amyloid A (SAA) into AA-amyloid, thus mimicking in part, or more preferably mimicking in its entirety, the process of amyloidogenesis observed in vivo. As demonstrated herein, this cell culture system is useful in identifying molecular interactions critical to amyloidogenesis. For example, using this cell culture system, the inventors have verified heparan sulfate to be an integral component of amyloid fibrils, and amyloid polypeptide:heparan sulfate interactions to be critical to amyloidogenesis. Further, this cell culture system is useful in identifying specific compounds that modulate these molecular interactions and/or amyloidogenesis. For example, the inventors have now identified a peptide-based compound that blocks amyloid deposition, specifically at a concentration that is several orders of magnitude lower than any other inhibitors previously reported. Accordingly, the present invention also relates to methods for identifying compounds that modulate amyloidogenesis and methods for identifying molecular interactions for targeting by compounds that will modulate amyloidogenesis. Further, the present invention relates to compounds which modulate amyloidogenesis and use of these compounds in treatment of amyloid-associated diseases including, but not limited to, Alzheimer's disease, familial polyneuropathy, spongiform encephalopathies (prion disorders such as scrapie and Creutzfeldt-Jakob disease), type II diabetes, and amyloid that occurs secondarily to lymphoma, chronic renal dialysis and rheumatoid arthritis.

### BACKGROUND OF THE INVENTION

Amyloids are complex tissue deposits composed of specific polypeptides and proteoglycans that accumulate in certain tissues thereby disrupting their architecture and function (Sipe, J. D. Clin. Lab. Sci. 1994 31:325-354; Sipe, J. D. and Cohen, A. S. J. Struct. Biol. 2000 130:88-98; Ancsin, J. B. Amyloid 2003 10:67-79). Amyloid can accompany or cause a wide range of medical conditions affecting millions of people, including Alzheimer's disease, familial polyneuropathy, spongiform encephalopathies (prion disorders such as scrapie and Creutzfeldt-Jakob disease), type II diabetes, lymphoma, chronic renal dialysis and rheumatoid arthritis. Each type of amyloid is identified by one of over 20 naturally occurring polypeptides which, in a poorly understood process, become re-folded into non-native conformational intermediates, and assemble into fibrils of a highly regular structure. Despite the diversity of amyloid precursor polypeptides and the associated diseases, all amyloid fibrils purified from tissue are composed of several 3 nm filaments (proto-fibrils) that are twisted around each other in a shallow helix forming non-branching fibrils of 7-10 nm in diameter. The polypeptides are arranged in a cross-β-pleated sheet conformation that is oriented perpendicular to the longitudinal axis of the fibrils. Amyloids stain with Congo Red (CR) and when viewed under polarized light exhibit a red-green birefringence, a property considered diagnostic for amyloid.

Serum amyloid A (SAA), an acute-phase apoprotein of high density lipoprotein (HDL), was one of the first amyloidogenic proteins discovered, producing AA-amyloid in a patient with persistent acute inflammatory diseases (Benditt et al. FEBS Lett. 1971 19:169-173). Also first described for AA-amyloidosis and later substantiated in vitro with Aβ (the amyloid precursor of Alzheimer's disease) and prion amyloid polypeptides, was the observation that fibrillogenesis follows a nucleation-dependent mechanism (Axelrad et al. Lab. Invest. 1982 47:139-146; Jarrett, J. T. and Lansbury, P. T. Cell 1993 73:1055-1058; Harper, J. D. and Lansbury, P. T. Annu. Rev. Biochem,. 1997 66:385-407). The initial nucleation step is rate-limiting, during which a nucleus or “seed” is formed. The addition of a small amount of synthetic pre-formed fibril, or amyloid enhancing factor (AEF, an amyloid-tissue extract) eliminates this lag phase and initiates fibril formation (Axelrad et al. Lab. Invest. 1982 47:139-146; Jarrett, J. T. and Lansbury, P. T. Cell 1993 73:1055-1058; Harper, J. D. and Lansbury, P. T. Annu. Rev. Biochem. 1997 66:385-407).

Heparan sulfate, a glycosaminoglycan (GAG) found ubiquitously on cell surfaces and in the extracellular matrix, has been shown to co-deposit both temporally and spatially with the AA-fibrils in the spleen (Snow et al. Lab. Invest. 1987 56:665-675; Snow et al. J. Histochem. Cytochem. 1991 39:1321-1330). Examination of different types of amyloids, including Aβ (Snow et al. Am. J. Pathol. 1988 133:456-463; Perlmutter et al. Brain Res. 1990 508:13-19), AL (immunoglobulin light chain deposits; Young et al. Acta Neuropathol. (Berl) 1989 78:202-209), TTR (transthyretin; familial amyloidotic polyneuropathy; Magnus et al. Scand. J. Immunol. 1991 34:63-69), Cystatin C (hereditary cerebral hemorrhage; van Duinen et al. Lab. Invest. 1995 73:183-189), IAPP (islet amyloid polypeptide seen in 95% of type-II diabetes; Young et al. Arch. Pathol. Lab. Med. 1992 116:951-954) and PrP^{Sc} (prion disease; Snow et al. Lab. Invest. 1990 63:601-611), revealed that heparan sulfate is a universal component of amyloid in situ. Further, several studies have indicated that heparan sulfate plays a mechanistic role in amyloidogenesis. Heparan sulfate and no other GAG can increase the β-sheet content of murine SAA1.1, leaving the non-amyloidogenic 2.1 isoform unaffected (McCubbin et al. Biochem. J. 1988 256:775-783). A heparan sulfate-dependent shift in structure from random coil to β-sheet has also been observed for Aβ (Fraser et al. J. Neurochem. 1992 59:1531-1540) which precedes its rapid assembly into fibrils (McLaurin et al. Eur. J. Biochem. 1999 266:1101-1110). The ability of heparan sulfate to promote fibrillogenesis in vitro has also been reported for IAPP (Castillo et al. Diabetes 1998 47:612-620), α-synuclein (generates Lewy bodies in Parkinson's disease; Cohlberg et al. Biochemistry 2002 41:1502-1511) and phosphorylated tau protein, which forms the amyloid-like paired helical-filaments of neurofibrillary tangles in Alzheimer's disease (Goedert et al. Nature 1996 383:550-553). It has been suggested that the amyloid-promoting activity of heparan sulfate is facilitated through specific amyloid polypeptide: heparan sulfate interactions via binding sites which have been identified in Aβ (Narindrasorasak et al. J. Biol. Chem. 1991 266:12878-12883; Brunden et al. J. Neurochem. 1993 61:2147-2154), prion protein (Caughey et al. J. Virol. 1994 68:2135-2141; Warner et al. J. Biol. Chem. 2002 277:18421-18430), IAPP (Park, K. and Verchere, C. B. J. Biol. Chem. 2001 276:16611-16616), β-2-microglobulin (amyloid associated with chronic renal dialysis; Ohashi et al. Nephron 2002 90:158-168; Heergaard et al. J. Biol. Chem. 2002 277:11184-11189), immunoglobulin light chain (Jiang et al. Biochemistry 1997 36:13187-13194) and SAA (Ancsin, J. B. and S Kisilevsky, R. J. Biol. Chem. 1999 274:7172-7181).

U.S. Pat. No. 5,643,562 (Kisilevsky et al.), U.S. Pat. No. 5,728,375 (Kisilevsky et al.), U.S. Pat. No. 5,840,294 (Kisilevsky et al.), and U.S. Pat. No. 5,972,328 (Kisilevsky et al.) disclose therapeutic compounds and methods for inhibiting amyloid deposition. These compounds comprise an anionic group and a carrier molecule, or a pharmaceutically acceptable salt thereof and inhibit the interaction between amyloidogenic proteins such as (SAA) protein or beta-amyloid precursor protein and a glycoprotein or a proteoglycan constituent of a basement membrane including laminin, collagen type IV, fibronectin and heparan sulfate proteoglycan (HSPG), mimicking and/or competitively inhibiting the proteoglycan constituent. Arresting amyloidosis in vivo using small molecule anionic sulfonates or sulfates is also described by Kisilevsky et al. (Nature Medicine 1995 1(2) 143-148).

A peptide with a short amyloid beta-peptide fragment, KLVFF (SEQ ID NO:1), has been shown in vitro to bind full length amyloid beta-peptide and prevent its assembly into amyloid fibrils (Tjernberg et al. J. Biol. Chem. 1996 271(15):8545-8). This peptide fragment is suggested to serve as a lead compound in the development of peptide and non-peptide agents aimed at inhibiting amyloid beta-peptide in vi vo. Further, screening of combinatorial pentapeptide libraries composed of D-amino acids has led to identification of several ligands with a general motif containing phenylalanine in the second position and leucine in the third position, also capable in vitro of binding amyloid beta-peptide and preventing formation of amyloid like fibrils (Tjernberg et al. J. Biol. Chem. 1997 272(19):12601-5).

Peptide fragments corresponding to SNNFGA (residues 20-25; SEQ ID NO:2) and GAILSST (residues 24-29; SEQ ID NO:3) have also been disclosed as strong inhibitors in vitro of the beta-sheet transition and amyloid aggregation of human islet amyloid polypeptide, a major component of amyloid deposits found in the pancreas of patients with type-2 diabetes (Scrocchi et al. J. Mol. Biol. 2002 318(3):697-706). In addition, small peptides containing an HHQK (SEQ ID NO:11) domain of beta-amyloid inhibited plaque induction of neurotoxicity in human microglia (Giulian et al. J. Biol. Chem. 1998 273 (45) 29719-29726).

Intracellular cholesterol compartmentalization has also been linked to the generation of amyloid-beta peptide and ACAT inhibitors, developed for treatment of atherosclerosis, have been suggested to have potential use in the treatment of Alzheimer's disease (Puglielli et al. Nature Cell Biol. 2001 3:905-912).

### SUMMARY OF THE INVENTION

An aspect of the present invention relates to a cell culture system for amyloidogenesis. This cell culture system has been used by the inventors to verify heparan sulfate to be an integral component of amyloid fibrils, and amyloid polypeptide:heparan sulfate interactions as being critical to amyloidogenesis. Further, the inventors used this cell culture system to efficiently screen a number of compounds for their ability to modulate amyloid formation in the cells. Using this assay, compounds which inhibit amyloid formation and/or promote amyloid formation have been identified.

Accordingly, another aspect of the present invention relates to compounds which modulate amyloid formation. Preferably, the compounds of the present invention modulate the interaction of amyloid polypeptide with heparan sulfate by mimicking and/or competitively inhibiting binding of the amyloid polypeptide to the heparan sulfate. Additionally or alternatively, compounds of the present invention may bind to a cell surface receptor, thereby rendering the cell amyloid-resistant. Exemplary compounds identified herein with the capability to modulate amyloid formation include, but are in no way limited to, an isolated peptide ADQEANRHGRSGKDPNYYRPPGLPAKY (SEQ ID NO:6), also referred to as 27-mer peptide, corresponding to residues 77 through 103 of murine SAA1.1 and comprising a heparan sulfate binding site of murine SAA1.1, an isolated peptide ADQAANEWGRSGKDPNHFRPAGLPEKY (SEQ ID NO:9) corresponding to residues 78 through 104 of human SAA1.1, and a synthetic peptide ANRHGRSGKNPNYYRPPGLPAKY (SEQ ID NO:10), each of which is a potent inhibitor of amyloidogenesis. Also identified using this cell culture system was an isolated peptide WRAYTDMKEAGWKDGDKYFHARGNYDAAQRGPG (SEQ ID NO:7), also referred to as 33-mer peptide, corresponding to residues 17-49 of murine SAA1.1, which increases amyloid load in this cell culture system. These isolated peptides, as well as fragments, variants and mimetics thereof, are useful in modulating amyloid formation and amyloidogenesis. Further, it is expected that isolated peptides comprising the heparan sulfate binding sequence of SAA2.1 of these species, SAA1.1 and SAA2.1 from other species or the heparan sulfate binding sequence of other amyloid polypeptides such as Aβ or IAPP, as well as fragments, variants or mimetics thereof will serve as useful anti-amyloid agents.

Another aspect of the present invention relates to pharmaceutical compositions for modulating amyloid formation. The pharmaceutical compositions comprise a compound which modulates the interaction of amyloid polypeptide with heparan sulfate by mimicking and/or competitively inhibiting binding of the amyloid polypeptide to the heparan sulfate and/or binding to a cell surface receptor, thereby rendering the cell amyloid-resistant. These pharmaceutical compositions further comprise a pharmaceutically acceptable vehicle for in vivo administration of the compound.

Another aspect of the present invention relates to modulating cellular interaction of an amyloid polypeptide with heparan sulfate by administering to the cells a compound that mimics and/or competitively inhibits binding of the amyloid polypeptide via its heparan sulfate binding site and/or binds to a cell surface receptor thus rendering the cell amyloid-resistant. Modulating the interaction of an amyloid polypeptide with heparan sulfate using such compounds is useful in treating amyloid-associated diseases. Thus, such compounds are expected to be useful in the treatment of amyloid-associated diseases including, but not limited to, Alzheimer's disease, familial polyneuropathy, spongiform encephalopathies (prion disorders such as scrapie and Creutzfeldt-Jakob disease), type II diabetes, and amyloid that occurs secondarily to lymphoma, chronic renal dialysis and rheumatoid arthritis.

Another aspect of the present invention relates to a method for designing and/or identifying an anti-amyloidogenic agent by determining its ability to bind to and inhibit the amyloid enhancing activity of WRAYTDMKEAGWKDGDKYFHARGNYDAAQRGPG (SEQ ID NO:7) or a mimetic or fragment thereof.

### BRIEF DESCRIPTION OF THE FIGURES

FIG. 1 is a line graph showing similar kinetics of amyloidogenesis as determined by Thioflavin-T (Th-T) fluorescence between the cell culture system of the present invention pulsed with amyloid enhancing factor (AEF; filled circles) and mouse spleen (filled squares). Cells incubated with HDL-SAA alone, without the AEF pulse, experienced a lag-phase before the appearance of detectible amyloid (open circles).

FIG. 2A is a bar graph showing SAA isoform preference and the effect of SAA delipidation on AA-amyloidogenesis. Cells were incubated with delipidated SAA1.1, SAA2.1, HDL-SAA, reconstituted HDL-SAA1.1 and HDL-SAA2.1, and amyloid loads were assayed by Th-T fluorescence.

FIG. 2B provides a western blot evidencing that proteolytic processing of SAA1.1 in the cell culture system of the present invention is identical to that in amyloid-containing spleens.

FIG. 3 provides a line graph showing inhibition of amyloidogenesis in the cell culture system of the present invention by natural and synthetic anionic polymers. Cells undergoing amyloidogenesis were incubated with increasing concentrations of native-heparin (filled inverted triangles), low molecular weight heparin (LMW-Heparin, 3000 kD; filled triangles), chondroitin sulfate (filled diamonds) and polyvinyl sulfonate (PVS; filled squares) and the amyloid produced at the end of the protocol was assayed by Th-T fluorescence.

FIG. 4A is a line graph of competition curves (on a linear scale with respect to inhibitor concentration) comparing the ability of a 27-mer peptide of the present invention (filled circles) and a randomized 27-mer peptide (filled squares) to inhibit amyloidogenesis in the cell culture system. Also shown are competition curves (on a logarithmic scale with respect to inhibitor concentration) and determined IC₅₀s comparing the ability of the 27-mer peptide of the present invention (filled circles) and PVS (open circles) to inhibit amyloidogenesis in the cell culture system.

FIG. 4B provides a comparison of western blots showing that 50 μM LMW-heparin and PVS prevented HDL-SAA binding to J774 cells while the same concentration of the 27-mer peptide of the present invention did not.

FIG. 5A is a line graph comparing the ability of the 33-mer peptide in the presence of AEF (filled circles), the 33-mer in the absence of AEF (filled triangles) and a random 33-mer in the absence of AEF (filled squares) to promote amyloidogenesis in J774 cells. The 33-mer peptide not only promoted amyloidogenesis but also demonstrated AEF activity.

FIG. 5B shows a western blot which demonstrates that the 33-mer peptide at a concentration of 50 μM completely blocked HDL-SAA binding/uptake by J774 cells.

### DETAILED DESCRIPTION OF THE INVENTION

Amyloidosis is oftentimes a fatal condition in humans and is associated with a wide range of diseases. Its cause remains unknown and there are no effective treatments currently available. To better understand the condition of amyloidosis and identify and/or develop treatments for this condition, more information is needed regarding molecular interactions and/or binding sites of molecules involved in amyloidogenesis.

Peritoneal cells and a transformed peritoneal-macrophage cell line (IC-21 cells) have been reported to produce AA-amyloid when cultured for up to two weeks with continuous treatment with AEF and bacterially-expressed delipidated SAA (Kluve-Beckerman et al. Am. J. Pathol. 1999 155:123-133).

In the present invention, a new cell culture system is provided for amyloidogenesis. This cell culture system has been modified as compared to the cell culture system described by Kluve-Beckerman et al. (Am. J. Pathol. 1999 155:123-133) to provide an improved, physiologically relevant assay useful in identifying molecular interactions and/or binding sites of molecules involved in amyloidogenesis, and identifying and/or developing treatments for diseases caused by or relating to amyloid formation. Accordingly, the cell culture system of the present invention mimics in part, or more preferably in its entirety, steps and/or processes and/or characteristics of amyloidogenesis in vivo. For example, using this cell culture system, the inventors have verified heparan sulfate to be an integral component of amyloid fibrils, and amyloid polypeptide:heparan sulfate interactions as critical to amyloidogenesis. Thus, by the phrase “mimicking the step and/or processes of amyloidogenesis in vivo” it is meant that the cell culture system produces amyloid in the same manner as amyloid is produced in vivo and/or exhibits the same kinetics of amyloid deposition as observed in vivo, the same dependency upon AEF and/or SAA1.1 for amyloid formation as observed in vivo, and/or the same inhibitory characteristics of amyloidogenesis by compounds such as PVS and agents that truncate heparan sulfate synthesis as observed in vivo.

Using this cell culture system, the inventors have now identified compounds capable of modulating amyloid formation, which are believed to be effective therapeutic agents for diseases relating to amyloid formation.

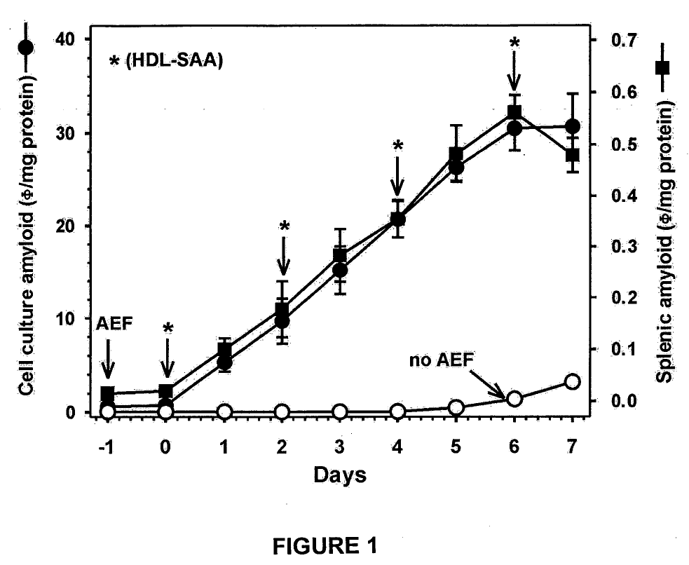

The cell culture system of the present invention preferably comprises a monocytic cell line or a tissue equivalent cell line comprising, for example, microglia or astrocytes from the brain, Kuppfer cells from the liver or reticuloendothelial cells from the spleen. Exemplary monocytic cells useful in the present invention include, but are not limited to, the murine monocytic cell line JM774A1 and the transformed peritoneal-macrophage cell line IC-21. The monocytic cells are cultured in a standard medium such as RPMI 1640 or DMEM containing 10 to 15% fetal bovine serum (FBS) for about 8 to about 10 days. Cells of the cell culture system of the present invention are then treated with physiological concentrations of native or reconstituted high density lipoprotein associated serum amyloid A (HDL-SAA) or synthetic micelles containing SAA1.1. HDL-SAA as well as the medium are replaced every other day, 3 to 4 times in total over the course of the protocol. As shown in FIG. 1, deposits of AA-amyloid were detectible in these cells by the end of the protocol, day 8. AA-amyloid deposits were detected by histochemical staining with Alcian blue and direct quantitation by Th-T fluorescence.

It has been found that amyloid load is proportional to the amount of HDL-SAA added up to about 0.3 mg/ml at which point amyloid load plateaus. Thus, while concentrations ranging from 0.05 mg/ml through 0.6 mg/ml of HDL-SAA result in detectible amyloid deposit formation in this cell culture system and accordingly can be used, a preferred concentration for both efficiency and economy is 0.3 mg/ml.

It is also preferred that on day 1 cells of the cell culture system of the present invention are treated with a pulse of an amyloid enhancing composition. This pulse preferably comprises treatment with a trace amount of the amyloid enhancing composition for at least 1 hour up to 24 hours with an amyloid enhancing composition. However, it is believed that pulse treatments shorter than 1 hour can also be used. Amyloidogenesis follows a nucleation-propagation mechanism and, as shown in FIG. 1, seeding with an exemplary amyloid enhancing composition, amyloid enhancing factor (AEF), eliminated the lag period observed in the same cell culture system treated with HDL-SAA alone. Various amyloid enhancing compositions for use in the cell culture system of the present invention are available. AEF, previously used in a mouse model for AA-amyloidosis (Kisilevsky et al. Lab. Invest. 1983 48:53-59) is demonstrated herein to be a useful amyloid enhancing composition in the cell culture system of the present invention. However, as will be understood by those of skill in the art based upon reading the instant application, other amyloid enhancing compositions, such as silk as described by Kisilevsky et al. (Amyloid 1999 6(2):98-106) and in Canadian Patent Application 2,251,427 published May 12, 1999, can be used.

It was found that treatment of the cells with a pulse of a trace amount of amyloid enhancing composition such as AEF was sufficient to enhance amyloid formation in the presence of native HDL-SAA. Further, incubation of cells with native HDL-SAA, instead of delipidated recombinant SAA as taught by Kluve-Beckerman et al. (Am. J. Pathol. 1999 155:123-133) resulted in 11 times more amyloid being produced. Similar amyloid formation is observed with reconstituted HDL-SAA and is expected with synthetic micelles containing SAA1.1. AEF, which contains amyloid fibrils, did not contribute to the CR staining or affect cell growth. However, as amyloid accumulated, cell numbers became reduced with the majority of the cells surrounding the extracellular amyloid deposits. Also, amyloidogenesis required viable cells, since fixation with formaldehyde prior to the amyloid induction protocol produced no amyloid.

Using Th-T fluorescence, the kinetics of amyloid deposition in cell culture and mouse spleens was monitored for 7 days post-AEF administration (see FIG. 1). Both exhibited the same kinetics, with amyloid being detected as early as 24 hours after either the addition of HDL-SAA in culture or the experimental induction of AA-amyloidosis in the mice. Amyloid deposition increased linearly for 6 days, at which time it appeared to plateau. Based on total protein content, amyloid deposition was about 54-fold greater in cell culture than in spleens. The absence of AEF in cell culture delayed the production of detectible amyloid by 5 days.

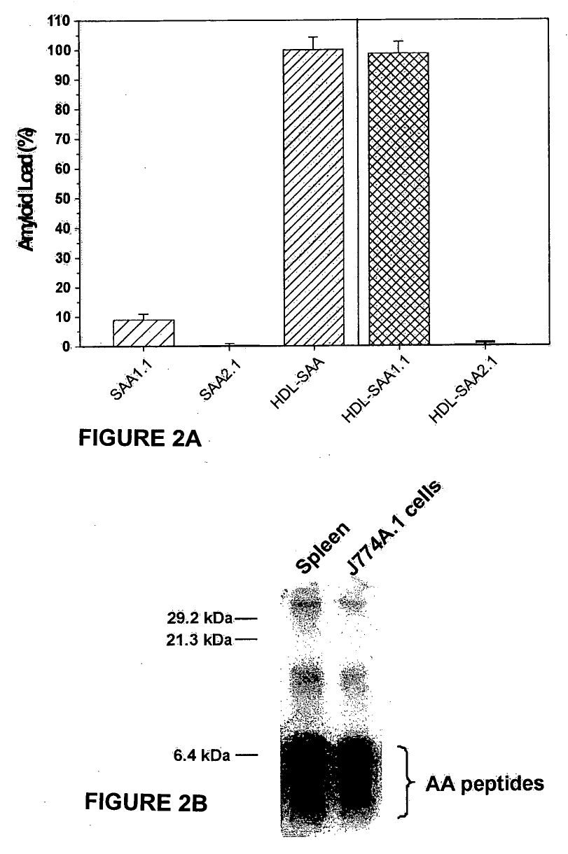

To determine which of the two major SAA isoforms was being converted into AA-amyloid in this assay system, cells were incubated as described above with AEF and either purified SAA1.1 or SAA2.1 at 50 μg/ml (which is equivalent to their respective concentrations in HDL-SAA at 0.3 mg/ml), HDL-SAA or HDL reconstituted with one or the other purified SAA isoform. The wells were then stained with CR for a qualitative assessment or assayed by Th-T fluorescence (See FIG. 2A). Only SAA1.1 produced amyloid, but in much reduced quantity (9% of HDL-SAA), while no amyloid was detected with SAA2.1. When purified SAA1.1 or 2.1 were first reassociated with HDL, the resulting amyloid load with the reconstituted HDL-SAA1.1 was close to the amount assayed for native HDL-SAA. Reconstituted HDL-SAA2.1 produced no amyloid. Thus, AA-amyloid formation detected in this cell culture system is derived from SAA1.1, as it is in vivo.

AA-amyloid fibrils are composed of a set of peptides spanning the amino-two-thirds of SAA1.1 (Benditt et al. FEBS Lett. 1971 19:169-173). Western blotting analysis using anti-SAA antibody showed that the proteolytic fragmentation of SAA1.1 appeared to be identical between cell culture and mouse amyloid-laden spleens (See FIG. 2B).

Of the six major GAGs (dermatan sulfate, chondroitin sulfate, keratan sulfate, heparin, heparan sulfate and hyauronan acid) only heparan sulfate has been shown to be a universal component of amyloids. Hence, the inventors determined which GAG, if any, was associated with the cell culture amyloid. Intense staining of cell culture amyloid was observed with Sulfated Alcian Blue (SAB), indicating a high sulfated GAG content. To distinguish between the different sulfated GAG species, wells containing amyloid were incubated with either a combination of heparanase/heparatinase, which specifically eliminates heparan sulfate, or chondroitinase ABC, which digests chondroitin and dermatan sulfate. Amyloid deposits treated with the heparan sulfate lyases showed no staining with SAB, although the residual amyloid deposits could still be discerned. Chondroitinase ABC-treated wells still exhibited strong SAB staining, further indicating that the majority of GAG associated with the amyloid fibrils was heparan sulfate.

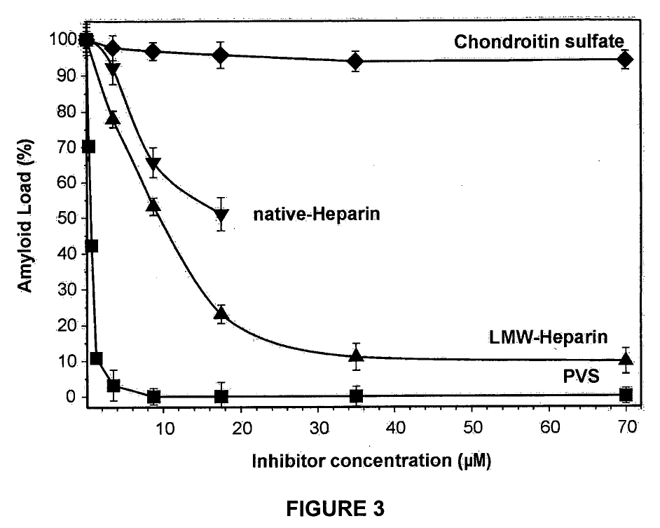

To examine whether sulfated GAGs are involved in the generation of the cell culture amyloid, the inventors tested the ability of native heparin (N-Heparin), low molecular weight heparin (LMW-Heparin) chondroitin sulfate and polyvinylsulfonate (PVS) to inhibit amyloidogenesis (FIG. 3). Clinically relevant doses of LMW-Heparin administered to mice undergoing AA-amyloidosis have been reported to reduce their amyloid load (Zhu et al. Mol. Med. 2001 7:517-522). It was found that both N-Heparin and LMW-Heparin inhibited the process of amyloidogenesis in cell culture. LMW-Heparin was well-tolerated by the cells but, at higher concentrations, N-Heparin became toxic. Chondroitin sulfate was unable to affect amyloid deposition at any concentration tested. PVS, a low molecular weight anionic polymer containing structural features similar to sulfated GAGs, achieved 50% and 100% inhibition (IC₅₀ and IC₁₀₀) at 0.5 μM and 9 μM, respectively. The anti-amyloid property of PVS has been demonstrated previously in vivo (Kisilevsky et al. Nat. Med. 1995 1:143-148).

Thus, as demonstrated by these experiments, the cell culture system of the present invention provides a model of amyloidogenesis correlating with in vivo amyloidogenesis. Further, this cell culture system provides an efficient assay for screening potential anti-amyloid compounds.

Accordingly, the present invention also provides a method for identifying potential anti-amyloid compounds using this cell culture system. In a preferred embodiment of this method, amyloidogenesis is induced in the cell culture by addition of an amyloid enhancing composition. HDL-SAA is then added. Typically a test agent is added after addition of the amyloid enhancing composition at the same time as HDL-SAA. However, inhibitory activity was also measured when test agents were added prior to addition of the amyloid enhancing composition. Thus, as will be understood by those of skill in the art upon reading this disclosure, test agents can be added prior to, in combination with, or subsequent to addition of the amyloid enhancing composition and/or the HDL-SAA.

The ability of a number of potential anti-amyloid compounds to modulate amyloid formation in this cell culture system was tested. To determine the effects of these potential anti-amyloid compounds, cell culture amyloidogenesis was induced in the presence of increasing concentrations of the test agents. Amyloid loads were quantified by Th-T fluorescence.

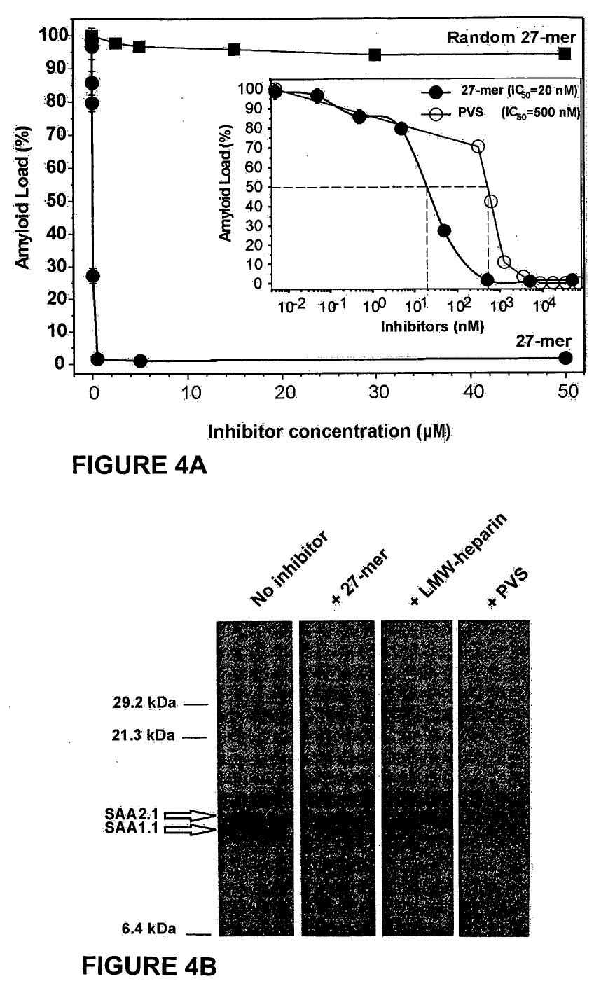

It was found that a synthetic peptide corresponding to SAA1.1's heparan sulfate binding site is highly anti-amyloidogenic. This heparan sulfate binding site, on the C-terminal end of murine SAA1.1 (77-ADQEANRHGRSGKDPNYYRPPGLPAKY-103; SEQ ID NO:6) was previously identified by Ancsin and Kisilevsky (J. Biol. Chem. 1999 274:7172-7181). As shown in FIG. 4A, this 27-mer peptide was a profound inhibitor of amyloidogenesis, with an ICSO of 0.02 μM, which is 25-fold lower than that for PVS. Further, this inhibitory effect was demonstrated to be sequence-specific, as scrambling the 27-mer sequence to produce a peptide PLPAQGKPGPDHYARNDSYAKNRYERG (SEQ ID NO:8), or replacing residues R83, H84 and R86 with A, which destroys heparan sulfate binding (Ancsin, J. B. and Kisilevsky, R. J. Biol. Chem. 1999 274:7172-7181), caused a complete loss of inhibitory activity. These experiments provide additional evidence of the activity of this 27-mer being sequence-specific and dependent on its basic, positively charged residues. Furthermore, this peptide did not interfere with HDL-SAA binding to cells, thus indicating that the amyloidogenic pathway was being affected specifically (see FIG. 4B). Unlike this peptide, both heparin and PVS prevented HDL-SAA binding to cells, which is likely responsible for their anti-amyloid activities. A synthetic peptide corresponding to residues 78-104 of human SAA1.1, ADQAANEWGRSGKDPNHFRPAGLPEKY (SEQ ID NO:9) demonstrated equivalent inhibitory activity.

A 27-mer peptide comprising D-amino acids (which are more stable in vivo) was also synthesized and its efficacy in the cell culture system was tested. At 20 μM, this peptide, when co-incubated with HDL-SAA, prevented the formation of any CR-detectable amyloid. The 27-mer peptide was also modified such that the amino-terminal 4 residues (which includes a D and E) were removed, the D90 was replaced with N, and the carboxyl-group at the carboxyl-terminus was amidated. This new derivative of the 27-mer, ANRHGRSGKNPNYYRPPGLPAKY (SEQ ID NO:10) which we refer to as the 23-mer basic, was able to completely inhibit CR-detectable amyloid at 2 μM. A summary of the peptides tested in the cell culture system of the present invention is shown in Table I.

TABLE I

(27-mer; SEQ ID NO:6)

77-ADQEANRHGRSGKDPNYYRPPGLPAKY-103

(27-mer; R83A/H84A/R86A; SEQ ID NO:20)

77-ADQEANAAGASGKDPNYYRPPGLPAKY-103

(D-27mer; SEQ ID NO:6))

77-ADQEANRHGRSGKDPNYYRPPGLPAKY-103

(23-mer basic; SEQ ID NO:10)

81-ANRHGRSGKNPNYYRPPGLPAKY-103

(h27-mer; SEQ ID NO:9)

78-ADQAANKWGRSGRDPNHFRPAGLPEKY-104

(33-mer; SEQ ID NO:7)

17-WRAYTDMKEAGWKDGDKYFHARGNYDAAQRGPG-49

(bold=substitutions from 27-mer SEQ ID NO:6; D=all residues are D-enantiomers; for the 23-mer basic the terminal carboxyl-group is amidated)

A number of other reports have described the ability of short peptides to inhibit Aβ and IAPP fibrillogenesis in vitro. In a cell-free system, an Aβ peptide (residues 16-20) at 100 μM, has been shown to associate with Aβ1-40 and prevent fibril assembly (Tjernberg et al. J. Biol. Chem. 1996 271(15):8545-8; Tjernberg et al. J. Biol. Chem. 1997 272(19):12601-5). Short IAPP peptides (residues 20-25 and 24-29), at a 10-fold molar excess (100 μM) over IAPP also reduced amyloid loads in vitro, by 80 to 85% (Scrocchi et al. J. Mol. Biol. 2002 318(3):697-706; Scrocchi et al. J. Struct. Biol. 2003 141(3):218-27). In the cell culture assay of the present invention, a similar level of inhibition could be achieved with 1400-fold less 27-mer (70 nM), which is about 60-fold less than the SAA1.1 concentration (4.2 μM) used to generate AA-amyloid in culture. Peptide fragments corresponding to LANFLV (residues 12-17; SEQ ID NO:4) and FLVHSS (residues 15-20; SEQ ID NO:5) of human islet amyloid polypeptide have been identified as strong enhancers of beta-sheet transition and fibril formation (Scrocchi et al. J. Struct. Biol. 2003 141(3):218-27). More recently, plasma cholesterol levels have been linked to cholesterol homeostasis in the brain and cholesterol lowering drugs as well as diet have been suggested to be valid candidates for the therapeutic treatment and prevention Alzheimer's disease (Puglielli et al. Nature Neuroscience April 2003 6(4):345-351).

A synthetic peptide, WRAYTDMKEAGWKDGDKYFHARGNYDAAQRGPG (SEQ ID NO:7), corresponding to residues 17-49 of murine SAA1.1, was demonstrated to have the opposite effect of increasing amyloid load in culture.

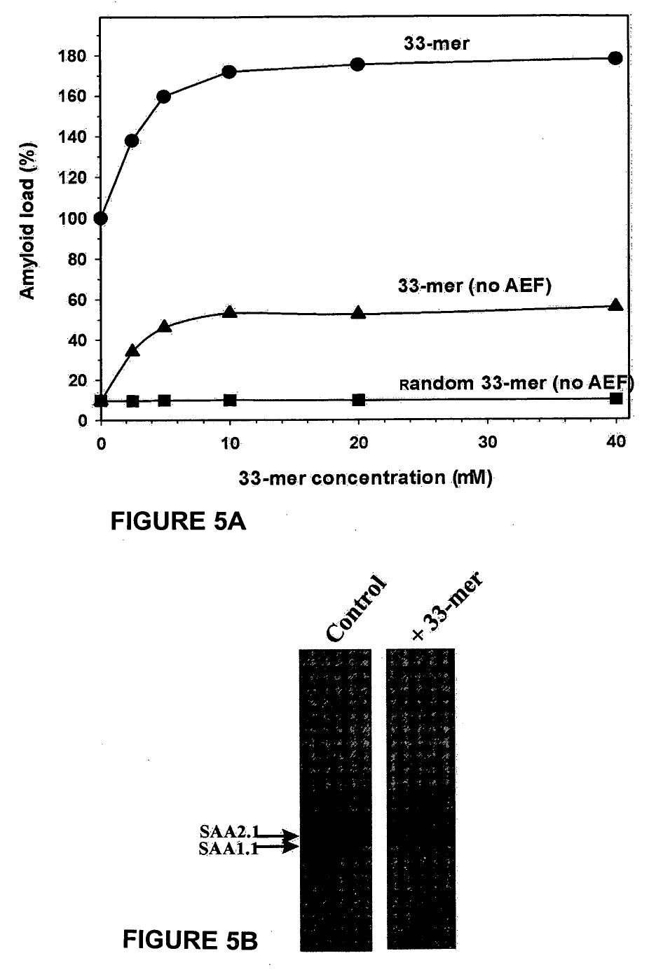

As shown in FIG. 5A, this 33-mer (SEQ ID NO:7) enhanced amyloid formation in J744 cells by up to 180% when the cells were preincubated with AEF, and by greater than 50% when AEF preincubation was not used. This enhancement of amyloid formation in the absence of AEF incubation is demonstrative of this 33-mer having intrinsic AEF activity. FIG. 5B shows that the mechanism of increased amyloid formation is through inhibition of acute phase HDL cell surface receptor binding, and/or intracellular uptake of acute phase HDL. Various receptors on the cell surface may potentially bind to acute phase HDL and promote its intracellular uptake. Some of the receptors responsible for this process may be the scavenger receptor or the FPRL1 receptor. Blockage of receptor binding and thus uptake of acute phase HDL (i.e. SAA1.1 HDL molecule) may be responsible for the promotion and formation of amyloid.

Identification of the 33-mer peptide increasing amyloid load is useful for the design and/or identification of agents that target this region of the amyloid polypeptide and that may inhibit the amyloidogenic activities of the amyloid polypeptide. In one embodiment, agents capable of inhibiting the amyloidogenic activity of this peptide are identified in the cell culture system of the present invention. In these experiments, the peptide of SEQ ID NO:7 is added to the cells of the culture. Test agents are also added and the ability of these agents to inhibit the increase in amyloid load in the cells caused by the peptide of SEQ ID NO:7 is determined.

Thus, the present invention also provides compounds which modulate amyloid formation or amyloidogenesis by mimicking an amyloid polypeptide, and more specifically the heparan sulfate binding site of an amyloid polypeptide or an amyloidogenic region of the amyloid polypeptide, thereby modulating binding and/or amyloidogenic activity of the amyloid polypeptide. Such compounds may also modulate amyloidogenesis by competitively inhibiting binding of the amyloid polypeptide to heparan sulfate or by binding to cell surface receptors, thus rendering the cells amyloid-resistant.

By the term “amyloid polypeptide” as used herein it is meant to be inclusive not only of SAA1.1 amyloid polypeptide but also amyloid polypeptides such as Aβ and IAPP as well as additional amyloid polypeptides as set forth in Table II, infra, and well known to those skilled in the art.

By the term “modulate”, “modulating, or “modulation” it is meant that a compound increases or decreases amyloid deposit formation in cell culture and/or in vivo. A compound of the present invention may modulate amyloid deposit formation by mimicking the amyloid polypeptide, or more preferably mimicking the heparan sulfate binding site of an amyloid polypeptide, or by competitively inhibiting binding of the amyloid polypeptide to heparan sulfate. Alternatively, compounds may modulate amyloid deposit formation by binding to a cell surface receptor, thereby rendering the cell amyloid-resistant.

Preferably, compounds are identified as modulators of amyloid formation in the cell culture system of the present invention.

Exemplary compounds of the present invention capable of modulating amyloid formation include, but are not limited to, the isolated peptide ADQEANRHGRSGKDPNYYRPPGLPAKY (SEQ ID NO:6) or a fragment, variant or mimetic thereof, the isolated peptide ADQAANEWGRSGKDPNHFRPAGLPEKY (SEQ ID NO:9), or a fragment, variant or mimetic thereof, the isolated peptide ANRHGRSGKNPNYYRPPGLPAKY (SEQ ID NO:10) or a fragment, variant or mimetic thereof, and the isolated peptide WRAYTDMKEAGWKDGDKYFHARGNYDAAQRGPG (SEQ ID NO:7), or a fragment, variant or mimetic thereof.

By “isolated” as used herein it is meant a peptide substantially separated from other cellular components that naturally accompany the native peptide or protein in its natural host cell. The term is meant to be inclusive of a peptide that has been removed from its naturally occurring environment, is not associated with all or a portion of a peptide or protein in which the “isolated peptide” is found in nature, is operatively linked to a peptide to which it is not linked or linked in a different manner in nature, does not occur in nature as part of a larger sequence or includes amino acids that are not found in nature. The term “isolated” also can be used in reference to synthetic peptides.

By synthetic peptides it is meant to be inclusive of recombinantly expressed peptides, chemically synthesized peptides, or peptide analogs that are biologically synthesized by heterologous systems.

Further, it will of course be understood, without the intention of being limited thereby, that a variety of substitutions of amino acids in the disclosed peptides is possible while preserving the structure responsible for the amyloid modulating activity. Conservative substitutions are described in the patent literature, as for example, in U.S. Pat. No. 5,264,558. It is thus expected, for example, that interchange among non-polar aliphatic neutral amino acids, glycine, alanine, proline, valine and isoleucine, would be possible. Likewise, substitutions among the polar aliphatic neutral amino acids, serine, threonine, methionine, asparagine and glutamine could possibly be made. Substitutions among the charged acidic amino acids, aspartic acid and glutamic acid, could possibly be made, as could substitutions among the charged basic amino acids, lysine and arginine. Substitutions among the aromatic amino acids, including phenylalanine, histidine, tryptophan and tyrosine would also likely be possible. In some situations, histidine and basic amino acids lysine and arginine may be substituted for each other. These sorts of substitutions and interchanges are well known to those skilled in the art. Other substitutions might well be possible. It is expected that the greater the percentage of sequence identity of a variant peptide with a peptide described herein, the greater the retention of biological activity. Variant peptides with substitutions which maintain the same polarity and distance between basic amino acids of the native peptide demonstrated to be required for binding to heparan sulfate are preferred. See U.S. Pat. No. 5,643,562. Peptide variants having the activity of modulating amyloid formation as described herein are encompassed within the scope of this invention.

By “fragment” or “fragments” it is meant to be inclusive of peptides exhibiting similar biological activities to the isolated peptides described herein but which, (1) comprise shorter portions of the anti-amyloid domain of murine or human SAA1.1 or the amyloid enhancing domain of murine SAA1.1 or (2) overlap with only part of the anti-amyloid domain of murine or human SAA1.1 or the amyloid enhancing domain of murine SAA1.1.

Further, the importance of the amyloid polypeptide:heparan sulfate interaction to amyloid formation identified herein, as well as the demonstrated inhibitory activity of peptides comprising SEQ ID NO:6, SEQ ID NO:10 and SEQ ID NO:9, are indicative of peptides comprising heparan sulfate binding sequences of SAA2.1 from murine and human and SAA1.1 and SAA2.1 from other species and peptides comprising heparan sulfate binding sequences from other amyloid polypeptides such as Aβ or IAPP, as well as fragments, variants or mimetics thereof, being useful anti-amyloid agents. While the amino acid sequences of heparan sulfate binding sites of various amyloids exhibit disparities in amino acid sequence, they share similarities in comprising a high percentage of basic residues spaced approximately 20 angstroms apart and exhibiting a positive charge overall. For example, heparan sulfate binding sites with these similar characteristics have been identified in other amyloid polypeptides that cause amyloids associated with Alzheimer's disease (A-β), prion disease (PrP^{sc}), diabetes (IAPP) and chronic renal dialysis (β-2-microglobulin). A summary table of these heparan sulfate binding sequences of various amyloid polypeptides is shown in Table II.

TABLE II

| | | |
|---|---|---|
| Aβ | 1-DAEFRHDSGYEVHHQKLVFFAEDVG | (SEQ ID NO:12) |
| | NKGIIGLMVGGVVIA-42 | |
| SAA1.1 | 17-WRAYTDMKEAGWKDGDKYFHARGN | (SEQ ID NO:7) |
| (mouse) | YDAAQRGPG-49 | |
| | 77-ADQEANRHGRSGKPNYYRPPGLPA | (SEQ ID NO:6) |
| | KY- 103 | |
| SAA1 | 78-ADQAANKWGRSGRDPNHFRPAGLP | (SEQ ID NO:9) |
| (human) | EKY- 104 | |
| proIAPP | 1-TPIESHQVEKRKCNTATCATQRLAN | (SEQ ID NO:13) |
| FLVHA-30 | | |
| β2m | 1-IQRTPKIQVYSRHPAENGKSN | (SEQ ID NO:14) |
| FLN-24 | | |
| PrP | 23-KKRPKPGGWNTGG-35 | (SEQ ID NO:15) |
| | 23-KKRPKPGGWNTGGSRYPGQGSPGG | (SEQ ID NO:16) |
| | NRYPPQ-52 | |
| | 53-GGGGWGQPHGGGWGQPHGGGWGQP | (SEQ ID NO:17) |
| | HGGGWGQPHGGGWGQGG-93 | |
| | 110-KHMAGAAAAGAVVGGLGGY-128 | (SEQ ID NO:18) |
| Tau | 317-KVTSKCGSLGNIHHKPGGG-335 | (SEQ ID NO:19) |
| protein | | |

Residues important to heparan sulfate binding appear in bold-face type (Ancsin, J. B. Amyloid 2003 10:67-79). Also see e.g. U.S. Pat. No. 5,643,562. These peptides, fragments, variants and mimetics thereof are also considered within the scope of the present invention.

By “mimetic” as used herein it is meant to be inclusive of peptides, which may be recombinant, and peptidomimetics, as well as small organic molecules, which exhibit similar or enhanced amyloid modulating activity. These include peptide variants which comprise conservative amino acid substitutions relative to the heparan sulfate binding peptide sequences of amyloid polypeptides, and peptide variants which have a high percentage of sequence identity with the native heparan sulfate binding sequences of amyloid polypeptides, at least e.g. 80%, 85%, 90%, and more preferably at least 95% sequence identity. Variant peptides can be aligned with the reference peptide to assess percentage sequence identity in accordance with any of the well-known techniques for alignment. For example, a variant peptide greater in length than a reference peptide is aligned with the reference peptide using any well known technique for alignment and percentage sequence identity is calculated over the length of the reference peptide, notwithstanding any additional amino acids of the variant peptide which may extend beyond the length of the reference peptide.

Preferred variants include, but are not limited to, peptides comprising one or more D amino acids, which may be equally effective but are less susceptible to degradation in vivo, and cyclic peptides. Cyclic peptides can be circularized by various means including but not limited to peptide bonds or depsicyclic terminal residues (i.e. a disulfide bond).

As used herein, the term “peptidomimetic” is intended to include peptide analogs that serve as appropriate substitutes for the peptides of SEQ ID NO:6, 7, 9 or 10 in modulating amyloid formation. The peptidomimetic must possess not only similar chemical properties, e.g. affinity, to these peptides, but also efficacy and function. That is, a peptidomimetic exhibits function(s) of an anti-amyloid domain of SAA1.1 or amyloid formation enhancing domain of SAA1.1, without restriction of structure. Peptidomimetics of the present invention, i.e. analogs of the anti-amyloid domain of SAA1.1 and/or the amyloid formation enhancing domain of SAA1.1, include amino acid residues or other moieties which provide the functional characteristics described herein. Peptidomimetics and methods for their preparation and use are described in Morgan et al. 1989, “Approaches to the discovery of non-peptide ligands for peptide receptors and peptidases,” In Annual Reports in Medicinal Chemistry (Vuirick, F. J. ed), Academic Press, San Diego, Calif., 243-253.

Mimetics of the present invention may be designed to have a similar structural shape to the anti-amyloid domain of SAA1.1 or the amyloid formation enhancing domain of SAA1.1. For example, mimetics of the anti-amyloid domain of SAA1.1 of the present invention can be designed to include a structure that mimics the heparan sulfate binding sequence. Mimetics of the anti-amyloid domain of SAA1.1 or the amyloid formation enhancing domain of SAA1.1 can also be designed to have a similar structure to the synthetic peptides of SEQ ID NO: 6, 9, 10 or 7, respectively. These peptidomimetics may comprise peptide sequences with conservative amino acid substitutions as compared to SEQ ID NO: 6, 9 or 10 or SEQ ID NO:7 which interact with surrounding amino acids to form a similar structure to these peptides. Conformationally-restricted moieties such as a tetrahydroisoquinoline moiety may also be substituted for a phenylalanine, while histidine bioisoteres may be substituted for histidine to decrease first pass clearance by biliary excretion. Peptidomimetics of the present invention may also comprise peptide backbone modifications. Analogues containing amide bond surrogates are frequently used to study aspects of peptide structure and function including, but not limited to, rotational freedom in the backbone, intra- and intermolecular hydrogen bond patterns, modifications to local and total polarity and hydrophobicity, and oral bioavailability. Examples of isosteric amide bond mimics include, but are not limited to, ψ[CH₂S], ψ[CH₂NH], ψ[CSNH₂], ψ[NHCO], ψ[COCH₂] and ψ[(E) or (Z)CH═CH].

Mimetics can also be designed with extended and/or additional amino acid sequence repeats as compared to the naturally occurring anti-amyloid domain of SAA1.1 and/or the amyloid formation enhancing domain of SAA1.1. Mimetics with such extensions, additions and/or repetitions of sequences may potentially increase efficacy as compared to the naturally occurring domain. Host cells can be genetically engineered to express such mimetics in accordance with routine procedures.

Identification of these peptide domains also permits molecular modeling based on these peptides for design, and subsequent synthesis, of small organic molecules that have amyloid modulating activities. These small organic molecules mimic the structure and/or activity of the peptides of SEQ ID NO:6, 7, 9 or 10. However, instead of comprising amino acids, these small organic molecules comprise bioisosteres thereof, substituents or groups that have chemical or physical similarities, and exhibit broadly similar biological activities.

Bioisosterism is a lead modification approach used by those skilled in the art of drug design and shown to be useful in attenuating toxicity and modifying activity of a lead compound such as SEQ ID NO:6, 7, 9 or 10. Bioisosteric approaches are discussed in detail in standard reference texts such as The Organic Chemistry of Drug Design and Drug Action (Silverman, R B, Academic Press, Inc. 1992 San Diego, Calif., pages 19-23). Classical bioisosteres comprise chemical groups with the same number of valence electrons but which may have a different number of atoms. Thus, for example, classical bioisosteres with univalent atoms and groups include, but are not limited to: CH₃, NH₂, OH, F and Cl; C₁, PH₂ and SH; Br and i-Pr; and I and t-Bu. Classical bioisosteres with bivalent atoms and groups include, but are not limited to: —CH₂— and NH; O, S, and Se; and COCH₂, CONHR, CO₂R and COSR. Classical bioisosteres with trivalent atoms and groups include, but are not limited to: CH═ and N═; and P═ and As═. Classical bioisosteres with tetravalent atoms include, but are not limited to: C and Si; and ═C⁺═, ═N⁺═ and ═P⁺═. Classical bioisosteres with ring equivalents include, but are not limited to: benzene and thiophene; benzene and pyridine; and tetrahydrofuran, tetrahydrothiophene, cyclopentane and pyrrolidine. Nonclassical bioisosteres still produce a similar biological activity, but do not have the same number of atoms and do not fit the electronic and steric rules of classical isosteres.

Additional bioisosteric interchanges useful in the design of small organic molecule mimetics of the present invention include ring-chain transformations.

Compounds of the present invention are preferably formulated into a pharmaceutical composition with a vehicle pharmaceutically acceptable for administration to a subject, preferably a human, in need thereof. Methods of formulation for such compositions are well known in the art and taught in standard reference texts such as Remington's Pharmaceutical Sciences, Mack Publishing Co., Easton, Pa., 1985.

An exemplary formulation demonstrated to be useful for many peptides is encapsulation of a compound in a phospholipid vesicle. An exemplary phospholipid vesicle which may be useful in the present invention is a liposome. Liposomes containing a compound of the present invention can be prepared in accordance with any of the well known methods such as described by Epstein et al. (Proc. Natl. Acad. Sci. USA 82: 3688-3692 (1985)), Hwang et al. (Proc. Natl. Acad. Sci. USA 77: 4030-4034 (1980)), EP 52,322, EP 36,676; EP 88,046; EP 143,949; EP 142,641; Japanese Pat. Appl. 83-118008, and EP 102,324, as well as U.S. Pat. Nos. 4,485,045 and 4,544,545, the contents of which are hereby incorporated by reference in their entirety. Preferred liposomes are of the small (about 200-800 Angstroms) unilamellar type in which the lipid content is greater than about 10 mol. percent cholesterol, preferably in a range of 10 to 40 mol. percent cholesterol, the selected proportion being adjusted for optimal peptide therapy. However, as will be understood by those of skill in the art upon reading this disclosure, phospholipid vesicles other than liposomes can also be used.

Pharmaceutical compositions of the present invention can be administered to a subject, preferably a human, to treat and/or prevent amyloid-associated diseases including, but not limited to, Alzheimer's disease, familial polyneuropathy, spongiform encephalopathies (prion disorders such as scrapie and Creutzfeldt-Jakob disease), type II diabetes, as well as amyloid that occurs secondarily to lymphoma, chronic renal dialysis and rheumatoid arthritis. The compositions may be administered by various routes including, but not limited to, orally, intravenously, intramuscularly, intraperitoneally, topically, rectally, dermally, sublingually, buccally, intranasallly or via inhalation. For at least oral administration, it may be preferred to administer a composition comprising a peptide with one or more D amino acids. The formulation and route of administration as well as the dose and frequency of administration can be selected routinely by those skilled in the art based upon the severity of the condition being treated, as well as patient-specific factors such as age, weight and the like.

The following nonlimiting examples are provided to further illustrate the present invention. The content of all references, pending patent applications and published patents cited throughout this application are hereby expressly incorporated by reference.

### EXAMPLES

### Example 1

### HDL-SAA and Purification of Delipidated SAA

Plasma HDL-SAA concentrations were experimentally elevated in CD1 mice (Charles River, Montreal, Quebec, Canada) by a subcutaneous injection of 0.5 ml of 2% (w/v) AgNO₃, as described by Ancsin and Kisilevsky (J. Biol. Chem. 1999 274:7172-7181), thereby producing a sterile abscess. After 18-20 hours, mice were sacrificed by CO₂ narcosis and exsanguinated by cardiac puncture. HDL-SAA was isolated by sequential density flotation in accordance with the procedure described by Havel et al. (J. Clin. Invest. 1955 34:1345-1353). SAA1.1 and 2.1 were isolated from HDL-SAA denatured with 6 M guanidine-HCl then purified by reversed phase-high performance liquid chromatography on a semi-preparative C-18 Vydac column connected to a Waters (Millipore) HPLC system (Ancsin, J. B. and Kisilevsky, R. J. Biol. Chem. 1999 274:7172-7181). Each isoform makes up about 16.7% of total HDL-SAA protein.

### Example 2

### Amyloid Enhancing Factor (AEF) Preparation

AEF was prepared as AA-amyloid fibrils in accordance with the procedure described by Axelrad et al. (Lab. Invest. 1982 47:139-146) and Kisilevsky et al. (Lab. Invest. 1983 48:53-59). For maximum activity, a 2 mg/ml stock of AEF was sonicated just before use. The AEF preparation was evaluated in a mouse model as described by Axelrad et al. (Lab. Invest. 1982 47:139-146) of AA-amyloidogenesis prior to use in cell culture.

### Example 3

### J774A.1 Cell Culture

The murine monocytic cell line J774A.1 (American Type Culture Collection, Manassas, Va.) was cultured in RPMI (Sigma) medium which contained 25 mM HEPES, 15% fetal bovine serum (FBS) and 50 μg/ml penicillin-streptomycin, at 37° C., 5% CO₂. The cell stocks were passaged every four days, and the medium replaced every other day. Cells were seeded at a minimal density in 8-well chamber slides (Lab-Tek®, Nalge Nunc International, Naperville, Ill.) in 350 μl medium/well and allowed to reach about 80-90% confluence (3 days), about 2.2×10⁵ cells per well. To induce AA-amyloidogenesis, cells were treated for 24 hours with 30 μg of AEF in the culture medium, then the medium was removed and the cells rinsed with fresh medium. To these cells 350 μl of medium was added containing either 0.3 mg/ml HDL-SAA, HDL, 0.05 mg/ml SAA1.1 or SAA2.1, replenished every two days for 7 days. At the end of the treatment period, the cells were either stained with Congo red to visualize the amyloid deposits, or the cells were dissolved in 1% NaOH and assayed for amyloid fibrils by thioflavin-T (Th-T) fluorescence as described by LeVine (Methods Enzymol. 1999 309:274-284). In some experiments, native heparin (Sigma), low molecular weight heparin (Sigma), chondroitin sulfate, polyvinylsulfonate or synthetic peptides were included at different concentrations throughout the HDL-SAA treatments. Some wells containing amyloid were digested with either 200 mU/well Chondroitinase ABC (Sigma), or 2 mU/well each of heparanase and heparatinase (Seikagaku America, Ijamsville, MD) in PBS, 2 mM CaCl₂ incubated for 4 hours at 37° C.

### Example 4

### Peritoneal Macrophages for Use in Cell Culture System

Peritoneal macrophages also develop amyloid in the culture system of the present invention. For these experiments, macrophages were harvested from mouse peritoneal cavity by lavage using RPMI medium. Cells were pelleted by centrifugation, re-suspended in RPMI+15% FBS and allowed to attach to the chamber slides. After the standard induction protocol as described in Example 3, amyloid was detectable by CR staining.

### Example 5

### Amyloid Detection and Quantitation

Congo Red (CR) staining for amyloid was performed on cells that were rinsed with PBS, fixed for 10 minutes in 70% ethanol, and then stained for 45 minutes with Congo red prepared in alkaline 80% ethanol, NaCl saturated solution. After counter-staining with Hematoxylin, slides were dehydrated with ethanol, washed with Citrisolv (Fisher) and prepared with Permount (Fisher) and a cover slip. Alcian Blue 8GX (0.45%) and sodium sulfate (0.45%) in 10% acetic acid (SAB) were used to stain sulfated polysaccharides followed by counter-staining with Van Giesen stain (1% acid fuchsin in 3% picric acid). Quantitation of amyloid was performed by Th-T fluorescence as described by LeVine (Methods Enzymol. 1999 309:274-284). Fluorescence spectra of Th-T were acquired at 25° C. with a Spectra Max Gemini 96-well plate reader. Cells were solubilized in 1% NaOH which was then neutralized (pH 7) and added to 6.25 μL of 2.5 mM Th-T (Sigma) in PBS. Control spectra of Th-T and cell extract alone were determined. The emission spectrum was collected by exciting the sample at 440 nm (slit width, 10 nm) and monitoring emission at 482 nm (slit width, 10 nm).

```
# SEQUENCE LIS
#TING
<160> NUMBER OF SEQ ID NOS: 20
<210> SEQ ID NO 1
<211> LENGTH: 5
<212> TYPE: PRT
<213> ORGANISM: Artificial sequence
<220> FEATURE:
<223> OTHER INFORMATION: Synthetic
<400> SEQUENCE: 1
Lys Leu Val Phe Phe
1 5
<210> SEQ ID NO 2
<211> LENGTH: 6
<212> TYPE: PRT
<213> ORGANISM: Artificial sequence
<220> FEATURE:
<223> OTHER INFORMATION: Synthetic
<400> SEQUENCE: 2
Ser Asn Asn Phe Gly Ala
1 5
<210> SEQ ID NO 3
<211> LENGTH: 7
<212> TYPE: PRT
<213> ORGANISM: Artificial sequence
<220> FEATURE:
<223> OTHER INFORMATION: Synthetic
<400> SEQUENCE: 3
Gly Ala Ile Leu Ser Ser Thr
1 5
<210> SEQ ID NO 4
<211> LENGTH: 6
<212> TYPE: PRT
<213> ORGANISM: Homo sapiens
<400> SEQUENCE: 4
Leu Ala Asn Phe Leu Val
1 5
<210> SEQ ID NO 5
<211> LENGTH: 6
<212> TYPE: PRT
<213> ORGANISM: Homo sapiens
<400> SEQUENCE: 5
Phe Leu Val His Ser Ser
1 5
<210> SEQ ID NO 6
<211> LENGTH: 27
<212> TYPE: PRT
<213> ORGANISM: Mus musculus
<400> SEQUENCE: 6
Ala Asp Gln Glu Ala Asn Arg His Gly Arg Se
#r Gly Lys Asp Pro Asn
1 5
# 10
# 15
Tyr Tyr Arg Pro Pro Gly Leu Pro Ala Lys Ty
#r
                        20
# 25
<210> SEQ ID NO 7
<211> LENGTH: 33
<212> TYPE: PRT
<213> ORGANISM: Mus musculus
<400> SEQUENCE: 7
Trp Arg Ala Tyr Thr Asp Met Lys Glu Ala Gl
#y Trp Lys Asp Gly Asp
1 5
# 10
# 15
Lys Tyr Phe His Ala Arg Gly Asn Tyr Asp Al
#a Ala Gln Arg Gly Pro
                        20
# 25
# 30
Gly
<210> SEQ ID NO 8
<211> LENGTH: 27
<212> TYPE: PRT
<213> ORGANISM: Artificial sequence
<220> FEATURE:
<223> OTHER INFORMATION: Synthetic
<400> SEQUENCE: 8
Pro Leu Pro Ala Gln Gly Lys Pro Gly Pro As
#p His Tyr Ala Arg Asn
1 5
# 10
# 15
Asp Ser Tyr Ala Lys Asn Arg Tyr Glu Arg Gl
#y
                        20
# 25
<210> SEQ ID NO 9
<211> LENGTH: 27
<212> TYPE: PRT
<213> ORGANISM: Homo sapiens
<400> SEQUENCE: 9
Ala Asp Gln Ala Ala Asn Glu Trp Gly Arg Se
#r Gly Lys Asp Pro Asn
1 5
# 10
# 15
His Phe Arg Pro Ala Gly Leu Pro Glu Lys Ty
#r
                        20
# 25
<210> SEQ ID NO 10
<211> LENGTH: 23
<212> TYPE: PRT
<213> ORGANISM: Artificial sequence
<220> FEATURE:
<223> OTHER INFORMATION: Synthetic
<400> SEQUENCE: 10
Ala Asn Arg His Gly Arg Ser Gly Lys Asn Pr
#o Asn Tyr Tyr Arg Pro
1 5
# 10
# 15
Pro Gly Leu Pro Ala Lys Tyr
                        20
<210> SEQ ID NO 11
<211> LENGTH: 4
<212> TYPE: PRT
<213> ORGANISM: Artificial sequence
<220> FEATURE:
<223> OTHER INFORMATION: Synthetic
<400> SEQUENCE: 11
His His Gln Lys
1
<210> SEQ ID NO 12
<211> LENGTH: 40
<212> TYPE: PRT
<213> ORGANISM: Homo sapiens
<400> SEQUENCE: 12
Asp Ala Glu Phe Arg His Asp Ser Gly Tyr Gl
#u Val His His Gln Lys
1 5
# 10
# 15
Leu Val Phe Phe Ala Glu Asp Val Gly Asn Ly
#s Gly Ile Ile Gly Leu
                        20
# 25
# 30
Met Val Gly Gly Val Val Ile Ala
                35
# 40
<210> SEQ ID NO 13
<211> LENGTH: 30
<212> TYPE: PRT
<213> ORGANISM: Homo sapiens
<400> SEQUENCE: 13
Thr Pro Ile Glu Ser His Gln Val Glu Lys Ar
#g Lys Cys Asn Thr Ala
1 5
# 10
# 15
Thr Cys Ala Thr Gln Arg Leu Ala Asn Phe Le
#u Val His Ala
                        20
# 25
# 30
<210> SEQ ID NO 14
<211> LENGTH: 24
<212> TYPE: PRT
<213> ORGANISM: Homo sapiens
<400> SEQUENCE: 14
Ile Gln Arg Thr Pro Lys Ile Gln Val Tyr Se
#r Arg His Pro Ala Glu
1 5
# 10
# 15
Asn Gly Lys Ser Asn Phe Leu Asn
                        20
<210> SEQ ID NO 15
<211> LENGTH: 13
<212> TYPE: PRT
<213> ORGANISM: Homo sapiens
<400> SEQUENCE: 15
Lys Lys Arg Pro Lys Pro Gly Gly Trp Asn Th
#r Gly Gly
1 5
# 10
<210> SEQ ID NO 16
<211> LENGTH: 30
<212> TYPE: PRT
<213> ORGANISM: Homo sapiens
<400> SEQUENCE: 16
Lys Lys Arg Pro Lys Pro Gly Gly Trp Asn Th
#r Gly Gly Ser Arg Tyr
1 5
# 10
# 15
Pro Gly Gln Gly Ser Pro Gly Gly Asn Arg Ty
#r Pro Pro Gln
                        20
# 25
# 30
<210> SEQ ID NO 17
<211> LENGTH: 41
<212> TYPE: PRT
<213> ORGANISM: Homo sapiens
<400> SEQUENCE: 17
Gly Gly Gly Gly Trp Gly Gln Pro His Gly Gl
#y Gly Trp Gly Gln Pro
1 5
# 10
# 15
His Gly Gly Gly Trp Gly Gln Pro His Gly Gl
#y Gly Trp Gly Gln Pro
                        20
# 25
# 30
His Gly Gly Gly Trp Gly Gln Gly Gly
                35
# 40
<210> SEQ ID NO 18
<211> LENGTH: 19
<212> TYPE: PRT
<213> ORGANISM: Homo sapiens
<400> SEQUENCE: 18
Lys His Met Ala Gly Ala Ala Ala Ala Gly Al
#a Val Val Gly Gly Leu
1 5
# 10
# 15
Gly Gly Tyr
<210> SEQ ID NO 19
<211> LENGTH: 19
<212> TYPE: PRT
<213> ORGANISM: Homo sapiens
<400> SEQUENCE: 19
Lys Val Thr Ser Lys Cys Gly Ser Leu Gly As
#n Ile His His Lys Pro
1 5
# 10
# 15
Gly Gly Gly
<210> SEQ ID NO 20
<211> LENGTH: 27
<212> TYPE: PRT
<213> ORGANISM: Artificial sequence
<220> FEATURE:
<223> OTHER INFORMATION: Synthetic
<400> SEQUENCE: 20
Ala Asp Gln Glu Ala Asn Ala Ala Gly Ala Se
#r Gly Lys Asp Pro Asn
1 5
# 10
# 15
Tyr Tyr Arg Pro Pro Gly Leu Pro Ala Lys Ty
#r
                        20
# 25
```

1. A cell culture system for amyloidogenesis comprising cells treated with physiological concentrations of native or reconstituted high density lipoprotein associated serum amyloid A (HDL-SAA) or synthetic micelles containing SAA1.1.

2. The cell culture system of claim 1 comprising monocytic cells.

3. The cell culture system of claim 1 further comprising a pulse of amyloid enhancing composition which is administered to the cells prior to treatment with HDL-SAA or synthetic micelles containing SAA1.1.

4. The cell culture system of claim 3 wherein the amyloid enhancing composition comprises amyloid enhancing factor.

5. The cell culture system of claim 1 which mimics amyloidogenesis in vivo.

6. A method for screening compounds for amyloid modulating activity comprising contacting the cell culture system of claim 1 with a test compound and comparing amyloid formation in cells of the culture system in the presence and absence of the compound, wherein a change in amyloid formation in the cells in the presence of the compound is indicative of the compound being a modulator of amyloid formation.

7. A pharmaceutical composition comprising a compound that mimics an amyloid polypeptide, competitively inhibits binding of an amyloid polypeptide to heparan sulfate or binds to a cell surface receptor, thereby rendering the cell amyloid-resistant and a pharmaceutically acceptable vehicle.

8. The pharmaceutical composition of claim 7 wherein the compound comprises an isolated peptide ADQEANRHGRSGKDPNYYRPPGLPAKY (SEQ ID NO:6) or a mimetic, variant or fragment thereof, an isolated peptide ADQAANEWGRSGKDPNHFRPAGLPEKY (SEQ ID NO:9) or a mimetic, variant or fragment thereof, or an isolated peptide ANRHGRSGKNPNYYRPPGLPAKY (SEQ ID NO:10) or a mimetic, variant or fragment thereof.

9. A method for modulating the interaction of an amyloid polypeptide with heparan sulfate in a subject comprising administering to the subject the pharmaceutical composition of claim 7.

10. The method of claim 9 wherein the pharmaceutical composition comprises an isolated peptide ADQEANRHGRSGKDPNYYRPPGLPAKY (SEQ ID NO:6) or a mimetic, variant or fragment thereof, an isolated peptide ADQAANEWGRSGKDPNHFRPAGLPEKY (SEQ ID NO:9) or a mimetic, variant or fragment thereof, or an isolated peptide ANRHGRSGKNPNYYRPPGLPAKY (SEQ ID NO:10) or a mimetic, variant or fragment thereof.

11. A method for treating an amyloid-associated disease in a subject comprising administering to the subject the pharmaceutical composition of claim 7.

12. The method of claim 11 wherein the amyloid-associated disease is Alzheimer's disease, familial polyneuropathy, a spongiform encephalopathy, a prion disorder, or type II diabetes.

13. The method of claim 11 wherein the pharmaceutical composition comprises an isolated peptide ADQEANRHGRSGKDPNYYRPPGLPAKY (SEQ ID NO:6) or a mimetic, variant or fragment thereof, an isolated peptide ADQAANEWGRSGKDPNHFRPAGLPEKY (SEQ ID NO:9) or a mimetic, variant or fragment thereof, or an isolated peptide ANRHGRSGKNPNYYRPPGLPAKY (SEQ ID NO:10) or a mimetic, variant or fragment thereof.

14. A method for treating amyloid that occurs secondarily to lymphoma, chronic renal dialysis or rheumatoid arthritis in a subject comprising administering to the subject the pharmaceutical composition of claim 7.

15. The method of claim 14 wherein the pharmaceutical composition comprises an isolated peptide ADQEANRHGRSGKDPNYYRPPGLPAKY (SEQ ID NO:6) or a mimetic, variant or fragment thereof, an isolated peptide ADQAANEWGRSGKDPNHFRPAGLPEKY (SEQ ID NO:9) or a mimetic, variant or fragment thereof, or an isolated peptide ANRHGRSGKNPNYYRPPGLPAKY (SEQ ID NO:10) or a mimetic, variant or fragment thereof.

16. A method for designing and/or identifying an anti-amyloidogenic agent comprising determining the ability of an agent to bind to and inhibit the amyloid enhancing activity of WRAYTDMKEAGWKDGDKYFHARGNYDAAQRGPG (SEQ ID NO:7) or a mimetic or fragment thereof.
